(19) United States
(12) Patent Application Publication
(10) Pub. No.: US 2025/0221811 A1
(43) Pub. Date: Jul. 10, 2025

(54) ANCHORING SYSTEM FOR VASCULAR IMPLANTS

(71) Applicant: STENTAL GMBH, Lörrach (DE)

(72) Inventors: Milisav OBRADOVIC, Lörrach (DE); Aleksandar OBRADOVIC, Lörrach (DE); Dirk DITTMANN, Basel (CH); Daniel BAUMGARTNER, Rheinfelden (DE)

(73) Assignee: STENTAL GMBH, Lörrach (DE)

(21) Appl. No.: 18/838,425

(22) PCT Filed: Feb. 14, 2023
(86) PCT No.: PCT/EP2023/053628

§ 371 Date: Aug. 14, 2024

(30) Foreign Application Priority Data
Feb. 18, 2022 (DE) 10 2022 103 887.4

(51) Int. Cl. A61F 2/07

(52) U.S. Cl. CPC A61F 2/07; A61F 2002/072; A61F 2220/0008

(57) ABSTRACT

The invention relates to an anchoring system for vascular implants comprising a multitude of anchoring elements (4) in the form of threads and/or sutures formed from threads and/or knots (4a, 4b), which are provided on the inside (i) of the vascular implant (1). The anchoring system allows a reliable connection of several telescopically arranged vascular implants.

The invention relates to an anchoring system for vascular implants and vascular implants equipped with relevant anchoring systems.

A wide range of vascular diseases and vascular anomalies can nowadays be treated using minimally invasive techniques which involve the insertion of vascular implants. Depending on the nature of the disease or malformation and the state of the vessel to be treated, the attending physician has a wide range of different types of vascular implants at his or her disposal.

Aneurysms are one of the most common pathologies of the human vascular system. Aneurysms are protuberances or bulges occurring in the sectionally weakened vascular wall. Due to the pressure constantly acting on the weakened vessel wall the aneurysm will widen more and more, so that it may eventually rupture, leading to severe internal bleeding.

Basically, aneurysms may occur in any part of the blood vessel system. However, there are certain areas of the blood vessel system that are predestined for the formation of aneurysms because of their vascular anatomy, for example vessel bifurcations.

Aneurysms can result in problems if they press against other vessels or organs, for example. This may cause pain and/or result in an undersupply of other parts of the body and organs. If the brain is affected, for instance, more or less severe neurological deficits are the result.

In the event the aneurysm ruptures, this is particularly dangerous for the patient when the aneurysm is situated in the brain or on a large vessel carrying or conducting a great volume of blood. In the brain, ruptured aneurysms lead to cerebral hemorrhages, which may cause mild to most severe neurological damage. In the worst case, such a brain hemorrhage can lead to the death of a patient.

In case of a rupturing large vessel aneurysm, fatal blood loss can occur within a very short time, for example if this involves abdominal artery aneurysms or thoracic aneurysms.

Only a few years ago, many aneurysms had to be treated by open surgery. There is now a fairly wide range of alternative treatment options and medical devices available for the minimally invasive and intravascular treatment of aneurysms.

The placement of vascular implants in the form of vascular prostheses or stent grafts is one of the preferred minimally invasive, intravascular treatment options for aneurysms. A vascular prosthesis respectively stent graft is a type of vascular implant that is made up of a stent frame and a membranous coating that in combination produce a tubular implant. These are also referred to as covered implants or covered stents.

For the treatment of an aneurysm, such a covered implant bridges the area of the aneurysm in that the implant connects the healthy portions of the vessel proximally and distally of the aneurysm, thus resulting in the blood passing through the implant in the area of the aneurysm. As blood is no longer allowed to pass through the aneurysm, the aneurysm is prevented from rupturing and the pressure exerted by the aneurysm on other structures is largely eliminated.

In order to prevent that the secondary branches are cut off from the blood flow in the area of the covered implant, it is necessary to supply these side branches for example through conduits/leads of the covered implant. A multitude of techniques are known in the state of the art that offer possible solutions for this. Nowadays, it is primarily the custom-made or modular vascular prostheses that have established themselves, which are tailored to the needs of each individual patient. Such custom-made products and modular systems are expensive.

Manufacturing customized products in particular also is time consuming, as they usually have to be made individually by hand. This prevents the possibility of taking rapid action when needed. Modular systems as well as tailor-made implant systems are assembled by the surgeon within the patient's vascular system in the course of the intervention.

The drawback of such implants with prefabricated branches is the large amount of space they require in the non-expanded state, which makes it more difficult to use a catheter to place them in position, especially due to increased friction. In general, large-caliber catheters are required for the placement of such implants.

Concepts of meanwhile known alternative implants, which are discussed in more detail below, in particular the so-called multilumen implants, differ significantly from those described hereinafter and referred to as conventional implants. With the use of conventional implants, it is attempted to more or less accurately replicate the anatomy of the damaged vascular area with a view to providing a lining for the region to be rehabilitated or bridging an aneurysm. Accordingly and as described hereinbefore, these implants will either be individually fabricated or assembled in modular form.

On the other hand, a multilumen implant in principle is a kind of distributor insert provided with a proximal opening and as a rule several distal branches or junctions. A multilumen implant is placed in position proximal to the aneurysm, with its distal branches being individually connected via covered stents to the vessels located distal to the multilumen implant.

Such a multilumen implant is described, for example, in WO 2019/219728 A1.

The multilumen implant essentially consists of a hose-like or tubular element and an element by means of which the proximal area of the tubular element is secured in a vessel. The distal section of the multilumen implant preferably branches into at least two lumens. The multilumen implant can thus be thought of as a glove, with the distally located lumens representing the open-ended fingers.

Distal in this connection refers to the part of a structure that points in the direction of the blood flow and thus indicates the end of the implant, while proximal refers to the part that faces against the blood flow direction and thus constitutes the starting point of the implant. Further information on the location designations can also be found in the figures.

The described conventional implants and the multilumen implants have in common that they usually consist of several elements—as a rule different covered implants as well as stent grafts—that have to be assembled and anchored to each other in the vessel system.

Common to these well-known implants or implant systems is that they usually comprise a proximal main element, which may be a multilumen implant, and secondary elements which are connected to it distally and/or laterally and which as a rule are stent grafts.

In this context, it is crucial for the long-term lasting safety of the entire implant system that the individual elements are securely attached to or inside each other.

As a rule, the individual elements are attached telescopically by inserting a proximal section of the distal element into a distal section of the proximal element. Subsequently, at least the proximal section of the distal element is widened to such an extent that it forms a force-locked or friction-fit connection with the proximal element.

Regularly, the distal sections of the proximal element can be shaped so as to form special attachment points that allow for the most effective connection possible. For example, these sections are designed to have a distal taper, with distal stiffeners or combinations thereof, which enables the inserted elements to be firmly seated.

It has been observed here that, despite the provision of special attachment points, dislocations may still arise in the event the inserted elements become loose in the seat, shift or, in the worst case, even slip out of the connection.

Moreover, the disadvantage of known attachment points, in particular in the form of stiffeners, which as a rule consist of stent-like elements or rings, is that these stiffeners increase the volume of the relevant implant and, in particular, the diameter in the compressed state for delivery through a catheter, and they also result in increasing the stiffness of the compressed implant.

As a result, this makes it necessary to use large-caliber delivery catheters to enable the implant to be inserted, and, in addition, the implant's rigidity impairs maneuverability within the vascular system.

It is therefore the objective of the present invention to provide anchoring elements for vascular implants and in particular for covered vascular implants such as vascular prostheses for example, covered stents and multilumen implants, which do not suffer from the disadvantages previously described and which enable an improved fixation of distal and proximal sections in such a system.

This objective is achieved by an invention encompassing the characteristics of claim 1, with advantageous embodiments in each case being the subject of the dependent claims. It is to be noted that any features and characteristics individually included in the claims may also be combined with each other in an optional and technologically sensible manner so that they reveal further implementations or methods of the invention.

The anchoring system for vascular implants proposed by the invention comprises a plurality of anchoring elements in the form of threads and/or sutures formed from threads and/or knots formed from threads, which are provided at least partially on the inside of the vascular implant.

The inner lumen of the vascular implant represents the lumen of the vascular implant that is covered by the membrane or surrounded by a frame of a stent, with the inner side of the vascular implant denoting the side of the vascular implant that interfaces with the inner lumen. Accordingly, the sutures/seams and/or knots, of which a significant proportion is intended to be located on the inside of the vessel implant, are as a rule arranged on the inside of the membrane or the stent frame.

The term “thread” shall be understood to refer not exclusively to yarns and twists, but shall also denote filaments and fibers in a broad sense. Moreover, the material of an inventive thread shall not be restricted by the term definition, but may encompass substances of natural or artificial origin. The thread can be monofilament, that is, made of a single fiber, or polyfilament, that is, made of several fibers (for example, braided fibers, etc.).

However, the thread does not have to be made of fibers; in principle, it can also consist of fine wires or wire mesh made of metal.

An essential feature of the thread proposed by the invention is that it possesses the desired properties such as tensile strength and biocompatibility in order to provide a reliable and durable connection point.

Preferably, the sutures have loops that extend into the interior of the vascular implant and in this way create a particularly advantageous connection point for further vascular implants introduced there.

The knots, sutures and threads can be arranged symmetrically, for example in a ring, helix, spiral or screw shape, but may as well be provided in a random pattern. The ring-shaped knots, sutures and threads can be arranged in several layers, for example comprising several rings placed on top of each other, or arranged in one layer only.

Such a ring comprises at least three knots, however, preferably 4 to 16 and in particular 6 to 8 knots, which preferably are configured in a hexagonal shape in an equilateral hexagon or in an octagonal shape in an equilateral octagon.

Preferably, at least some of the knots are connected to each other by threads in such a manner that the threads pass through the lumen of the vascular implant and are not, as in the event of a suture, regularly secured along the membrane or the stent frame. Accordingly, the threads run preferably from one knot to the other knot freely through the lumen of the vascular implant.

The threads running between the knots are preferably taut and do not form loops. In this way, they serve as additional anchoring points for the insertion of another vascular implant in this area, and, moreover, due to their taut arrangement they do not impede the introduction of a further vascular implant, as they only reduce the opening of the lumen to a minor extent.

Preferably, neighboring knots are each connected by threads. The threads, in conjunction with the knots between which they run, and the corresponding section of the vessel implant form an arc in which the thread forms the chord, or, respectively, one side of the possibly resulting polygon, which may, for example, be a hexagon or octagon.

The connections between the knots can be correspondingly restricted to one level or, in addition or alternatively, may alternate between the levels, either one or more times in a zigzag pattern, for instance.

The anchoring system proposed by the present invention is particularly suitable for use in combination with the multilumen implant disclosed in WO 2019/219728 A1, for example. In this case, the inventive anchoring system can be provided primarily at the distal ends of the distal branches of the implant.

Even though the anchoring system proposed by the invention is described hereinafter mainly using the example of a multilumen implant, the system is suitable for vascular implants in general and for covered vascular implants in particular.

The known multilumen implant consists of an essentially hose-like or tubular element and a fixation element in the form of a stent or clamping ring by means of which the tubular element is secured in a vessel.

The tubular element is structured to form a proximal and a distal section, with the distal section branching into at least two lumens.

The tubular element is secured in the target vessel proximal to the aneurysm using one or more fixation elements. Taking further measures, the tubular element can be attached to one or several fixation rings, for example by clamping, welding, bonding, suturing or other known techniques. The fixation elements are located in the proximal section of the tubular element.

The distal section of the tubular element preferably branches into at least two branches, with the number of branches or bifurcations being arbitrary and the number of branches may be selected, for example, depending on the number of vessels to be perfused. Preferred are embodiments featuring two to five branches

The distal branches of the implant are usually connected to the vessels located distally to the implant via covered stents, so-called stent grafts, so that the aneurysm area is bridged, thus largely preventing the flow of blood into the aneurysm and relieving pressure acting on the aneurysm. At the same time, the distally located vessels are effectively supplied with blood via the stent grafts.

With a view to improving the fixation of the stent grafts, the anchoring system according to the invention can be provided in the distal areas of the implant, at least in some of the branches. The anchoring system is preferably provided on the membrane of the implant.

For this purpose, for example, the majority of the threads forming the knots and/or sutures are provided on the inside of the branches, i.e. in the lumen of the branches. Accordingly, this also applies to the loops that can be formed by the thread of the sutures.

The possible arrangements and combinations of the anchoring elements correspond to the basic disclosure of the invention.

The anchoring system according to the invention can also be provided in addition to existing elements, such as nitinol rings, stent structures or similar devices, or may encompass these elements.

The anchoring system proposed by the invention offers advantages over the state of the art in that the anchoring elements, which are made of threads, are significantly more flexible than known stent-like anchoring elements and are also stable and form strong and reliable anchoring points for further vascular implants.

Moreover, the inventive anchoring system comprising anchoring elements formed by threads, can be compressed far more easily than known stent-like anchoring elements. As a result, the anchoring system of the invention does neither excessively enlarge the diameter nor stiffen the compressed vascular implant, so that an impairment of the maneuverability of the vascular implant in the delivery catheter is not noticeable and the necessity of using larger-caliber delivery catheters does not arise.

The invention as well as the technical environment are described hereunder in sufficient detail on the basis of the figures. It is to be noted that the figures show an especially preferred embodiment variant of the invention. However, the invention shall not be deemed as being limited to the embodiment variant shown. To the extent it is technically expedient, the invention comprises, in particular, any optional combinations of the technical features that are stated in the claims or in the description as being relevant to the invention.

Clarification of the invention is provided by the following figures where

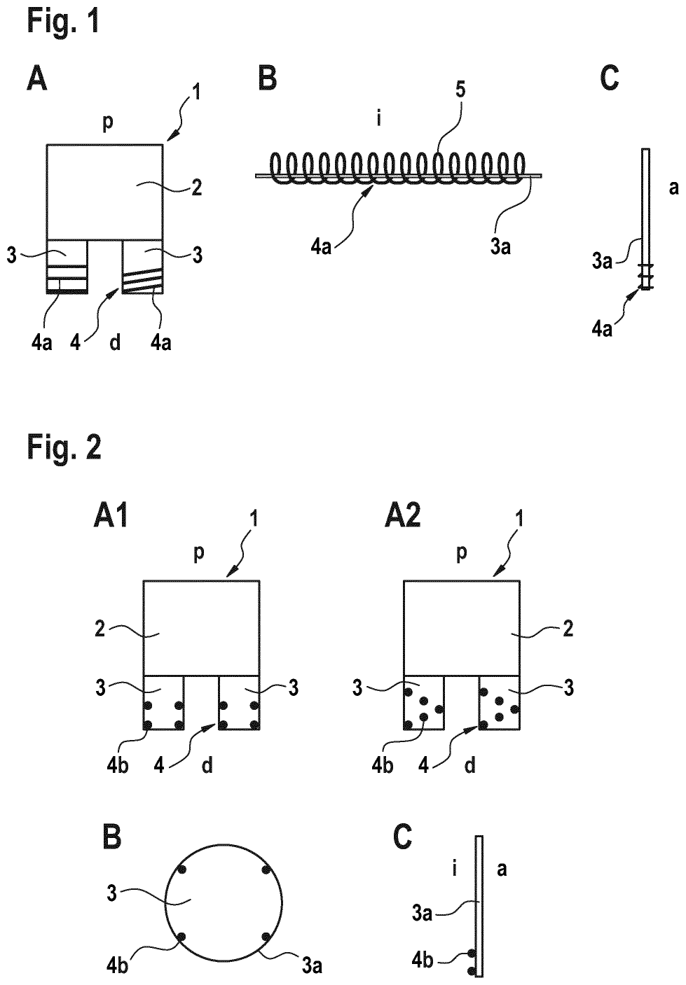

FIG. 1 shows a first embodiment of the anchoring elements in the form of ring- or helical-shaped sutures;

FIG. 2 illustrates a second embodiment of the anchoring elements in the form of knots of ring- or helical-shaped arrangement;

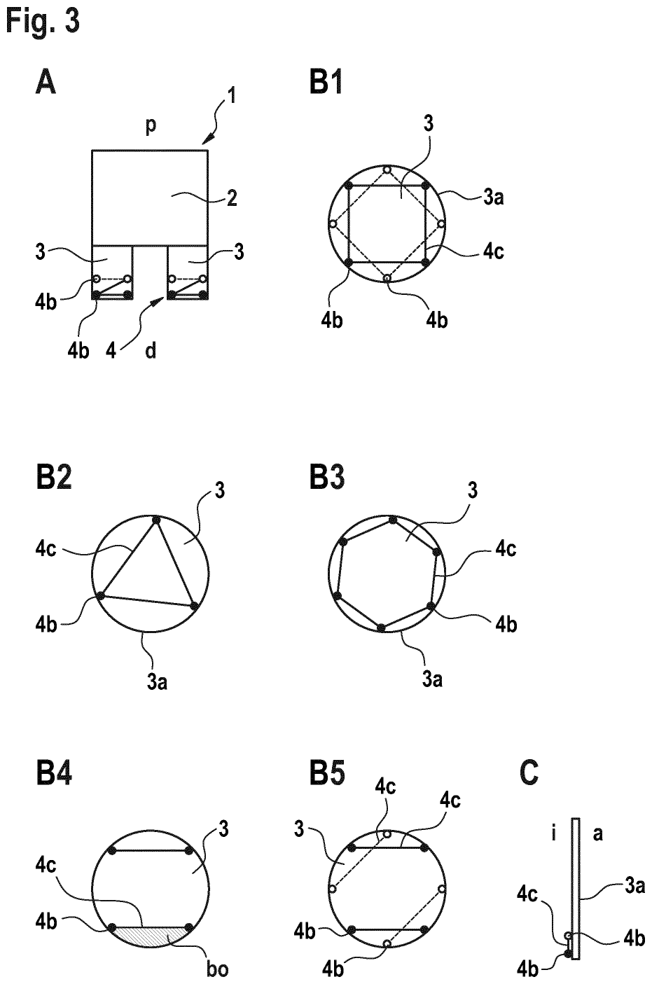

FIG. 3 shows a third embodiment of the anchoring elements in the form of a combination of ring-or helical-shaped sutures and knots;

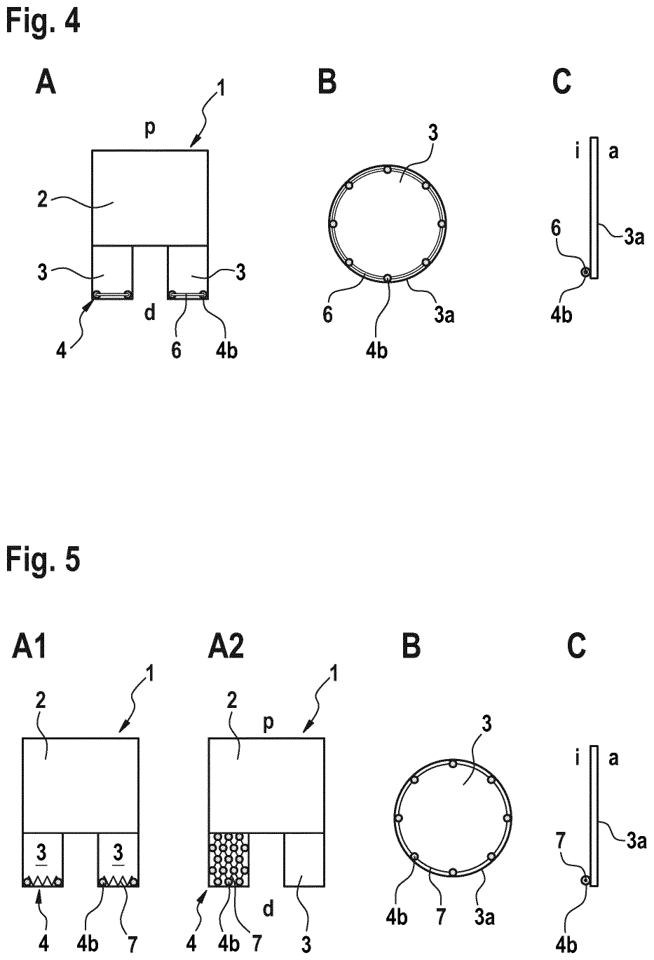

FIG. 4 depicts a combination of the anchoring elements according to the invention with known reinforcements made of nitinol rings;

FIG. 5 illustrates a further combination of the inventive anchoring elements with known reinforcements made of stent segments.

FIG. 1 shows in illustration A a first embodiment of the anchoring system provided by the invention with anchoring elements 4 in the form of ring-or helically arranged sutures 4a in connection with a multilumen implant 1, represented schematically only, comprising a main lumen 2 and two secondary lumens 3. The anchoring system 4 can also be provided as a seam consisting of a single circumferential suture 4a, for example at the proximal end of the secondary lumen 3.

The anchoring system can of course be suitably provided for a wide range of vascular implants, particularly covered vascular implants.

Sutures 4a thicken the membrane 3a in sections and stabilize it at the same time, as shown in illustration C. The ring-or helical-shaped sutures 4a serving as thickenings form anchor points for the fixation of an optionally inserted additional vascular implant (not shown), making it much less likely that the additional vascular implant will dislocate or slip out than without the corresponding anchoring elements 4.

Preferably and as shown in illustration B, the sutures 4a are provided in such a way that the portion of suture 4a positioned on the inside i of the membrane 3a at least partially forms loops 5 that serve as additional anchor points for an inserted vascular implant. The openings of loops 5 can be arranged at different angles, for example perpendicular or parallel to the opening of the vessel implant 1 or, respectively, the secondary lumens 3.

FIG. 2 shows a second embodiment of the anchoring system provided by the invention with anchoring elements 4 in the form of ring-or helically arranged knots 4b, again, as an example, in connection with a multilumen implant 1, represented schematically only, comprising a main lumen 2 and two secondary lumens 3.

As shown in illustrations B and C the knots 4b form individual thickenings, essentially situated on the inside i of the membrane 3a, which serve as anchor points for an inserted vascular implant (which is not shown here).

As shown in illustration A1, the knots 4b can be arranged in a ring or helically, as shown in illustration A2.

Aside from these two arrangements, a plurality of other symmetrical or also random arrangement patterns are of course conceivable, which may be adapted, if thought expedient, to the structure of the vessel implant to be inserted.

A third embodiment of the inventive anchoring system can be seen in FIG. 3, with anchoring elements 4 provided in the form of knots 4b of ring- or helical-shaped arrangement. The knots 4b can be arranged to form several levels, for example, the knots 4b constitute a first level or a first ring and further knots 4b form a second level or a second ring. Neighboring knots 4b are each connected preferably by a thread 4c that extends into the lumen of the secondary lumens 3.

The anchoring elements 4 are again shown as an example in conjunction with an only schematically depicted multilumen implant 1 consisting of a main lumen 2 and two secondary lumens 3.

Threads 4c protruding into the lumen are preferably stretched tightly between the knots 4a, 4b and in this manner form the main anchoring elements 4, in addition to the knots 4a, 4b. A tight tension, in contrast to a loose, loop-forming thread tension as shown, for example, in FIG. 1B offers advantages in this third embodiment in that an inserted further vascular implant (not shown) cannot become entangled in threads 4c, which might otherwise be swaying around uncontrollably in the bloodstream.

Preferably, this third embodiment comprises the arrangement of at least three knots 4b, which are interconnected by three threads 4c, as shown in illustration B2. A plurality of knots 4b and threads 4c, also arranged in several planes, is conceivable, as depicted in illustrations B1, B3 and C. The knots 4b are preferably arranged in a hexagonal or octagonal ring pattern.

Moreover, also conceivable are embodiments in which not all of the knots 4a, 4b are interconnected by threads 4c, but are only connected in pairs, for example, as can be seen in illustrations B4 and B5, with embodiment B5 similarly to B1 being provided with anchoring elements arranged in several planes.

An important aspect to distinguish this third embodiment from the embodiments featuring sutures, as shown for example in FIG. 1, is that the anchoring elements 4, which the third embodiment provides, do not form any loose loops and the threads 4c extend into the inside lumen i of the multilumen implant 1 in such a way that they form supporting elements for further vascular implants. Threads 4c in conjunction with the knots 4b between which they run and the associated section of the secondary lumen 3 form an arc bo, shown as an example in illustration B4. The 4c thread/threads form the chord of the arc or, in other words, the sides of the possibly resulting polygon, for example the hexagon as shown in illustration B3.

It is evident to those skilled in the art that this third embodiment does not have to follow the rather symmetrical arrangements in the examples, but that in this case as well the knots 4b and, as a result, the threads 4c can be arranged asymmetrically, and in this way create asymmetrical polygons.

It is also clearly evident to skilled persons that there can be combinations of the three above-mentioned embodiments in accordance with FIGS. 1 to 3.

FIG. 4 shows a first option for combining the inventive anchoring system including anchoring elements 4 with reinforcements consisting of nitinol rings 6, which are known for vascular implants—such as multilumen implants 1. The anchoring elements 4 in the form of knots 4b represent anchor points located on the inside i of the membrane 3a and, additionally, may be used and serve as connecting points between the membrane 3a and the nitinol ring 6. In addition or alternatively, the nitinol ring 6 can be embedded entirely or partially into the membrane 3a, with the distal part of the membrane 3a in this case being preferably folded inwards or outwards around the nitinol ring(s) 6. In such a configuration, the knots 4b can also be used to secure the folded membrane 3a.

Embodiments reflecting this are possible comprising a single or a plurality of nitinol rings 6 both in one as well as in several planes. Likewise, embodiments featuring a combination of several nitinol rings 6 are conceivable, with the individual rings 6 being of different diameter and causing the secondary lumens 3 to taper in distal direction, for example.

Knots 4b cause the roughness of the membrane inside i to increase thus preventing or at least make it more difficult for the optionally inserted vascular implant (not shown) to dislocate and, in particular, to slip out.

Moreover, knots 4b serve as additional fixation points for the nitinol ring 6 and the membrane 3a, thus providing additional safety in this area as well.

The knots 4b can be placed symmetrically or asymmetrically.

It is conceivably to combine the embodiment presented in FIG. 4 with additional anchoring elements 4 as depicted in FIGS. 1 to 3.

Similar to FIG. 4, FIG. 5 shows another option of connecting the anchoring system according to the invention with such reinforcements made of stent segments 7, which are known for vascular implants—such as multilumen implants 1.

The anchoring elements 4 in the form of knots 4b represent anchor points located on the inside i of the membrane 3a and, additionally, may be used and serve as connecting points between the membrane 3a and the stent segment 7. In addition or alternatively, the stent segment 7 can be embedded entirely or partially into the membrane 3a, with the distal part of the membrane 3a in this case being preferably folded inwards or outwards around the stent segment 7. In such a configuration, the knots 4b can also be used to secure the folded membrane 3a.

Relevant embodiments can be provided with a single-row stent segment 7 as shown in illustration A1 or a multi-row stent segment 7 as shown in illustration A2.

Knots 4b cause the roughness of the membrane inside i to increase thus preventing or at least make it more difficult for the optionally inserted vascular implant to dislocate and, in particular, to slip out.

Moreover, knots 4b serve as additional fixation points for the stent segments 7 and the membrane 3a, thus providing additional safety in this area as well.

The knots 4b can be placed symmetrically or asymmetrically.

It is conceivably to combine the embodiment presented in FIG. 5 with additional anchoring elements 4 as depicted in FIGS. 1 to 3.

A wide range of different vascular implants are known to those skilled in the art. For that reason, skilled persons are knowledgeable of the fact that the innovative anchoring system is suitable for a plurality of vascular implants and in particular for covered vascular implants. This applies in particular to the areas of a vascular implant where further vascular implants are to be inserted and secured which are as a rule the distal areas.

### List of Reference Numerals

1 Multilumen implant
2 Main lumen
3 Secondary lumens (3a: membrane)
4 Anchoring element (4a: suture, 4b: knot, 4c: thread)
5 Loop
6 Nitinol ring
7 Stent segment
bo Arc
i inside
a outside
p proximal
d distal

1. Anchoring system for vascular implants comprising a plurality of anchoring elements (4) in the form of threads and/or sutures and/or knots (4a, 4b) formed from threads, which are at least partially provided on the inside (i) of vascular implant (1).

2. Anchoring system for stent grafts according to claim 1, characterized in that the anchoring elements (4) are arranged in a ring-and/or helical-shaped configuration.

3. Anchoring system for vascular implants according to claim 1, characterized in that the sutures (4a) have loops (5) that extend into the lumen of the vascular implant (1).

4. Anchoring system for vascular implants according to claim 1, characterized in that the knots (4b) arranged in a ring form a polygon, in particular a hexagon or octagon.

5. Anchoring system for vascular implants according to claim 1, characterized in that at least some of the knots (4a, 4b) are connected to each other by threads (4c).

6. Anchoring system for vascular implants according to claim 5, characterized in that the threads (4c) pass through the lumen of the vascular implant (1) in such a way that, in conjunction with the knots (4a, 4b) between which they run and the associated section of the vascular implant (1), they form an arc (bo) with the thread (4c) forming the chord.

7. Vascular implant with an anchoring system (4) according to claim 1.

8. Vascular implant according to claim 7, wherein the vascular implant is a vascular implant covered with a membrane (3a).

9. Vascular implant according to claim 8, wherein the covered vascular implant is a multilumen implant having a main lumen (2) and at least two secondary lumens (3), and wherein the anchoring system (4) is arranged in the distal region of at least one of the secondary lumens (2).

10. Vascular implant according to claim 8, wherein the anchoring system (4) is provided on the membrane (3a).
